(19) United States
(12) Patent Application Publication
(10) Pub. No.: US 2026/0335479 A1
(43) Pub. Date: Oct. 29, 2026

(54) METHOD AND DEVICE FOR DISINFECTING AND/OR STERILISING ELECTRONIC COMPONENTS

(71) Applicant: SCHWING TECHNOLOGIES GMBH, Neukirchen-Vluyn (DE)

(72) Inventor: Alfred SCHILLERT, Dorsten (DE)

(21) Appl. No.: 19/117,008

(22) PCT Filed: Sep. 29, 2023
(86) PCT No.: PCT/EP2023/077147

§ 371 Date: Mar. 28, 2025

(30) Foreign Application Priority Data
Sep. 30, 2022 (DE) 10 2022 125 303.1

(51) Int. Cl. A61L 2/18; A61L 2/04; A61L 2/24; A61L 101/36; A61L 103/25

(52) U.S. Cl. CPC A61L 2/18; A61L 2/04; A61L 2/24; A61L 2101/36; A61L 2103/25; A61L 2202/122; A61L 2202/123; A61L 2202/13; A61L 2202/14; A61L 2202/15; A61L 2202/181

(57) ABSTRACT

The invention relates to a method for disinfecting and/or sterilizing electronic components, said method comprising the method steps of: placing an electronic component in a process chamber that can be evacuated; evacuating the process chamber; introducing a treatment medium; carrying out the disinfection and/or sterilization process; and increasing the pressure in the process chamber. The invention also relates to: a software program for carrying out the method for disinfecting and/or sterilizing electronic components; and a device for disinfecting and/or sterilizing electronic components. The invention also relates to: a disinfected electronic component comprising residues of the treatment medium and/or reaction products of the treatment medium and fewer than 2000 germs per m² on the surface of the disinfected electronic component; and a sterilized electronic component comprising residues of the treatment medium and/or reaction products of the treatment medium and a germ-free surface.

The invention relates to a method for disinfecting and/or sterilizing electronic components with the method steps of placing an electronic component in a process chamber that can be evacuated, evacuating the process chamber, introducing a treatment medium, carrying out the disinfection and/or sterilization process and increasing the pressure in the process chamber. The invention also relates to a software program for carrying out the method for disinfecting and/or sterilizing electronic components and to a device for disinfecting and/or sterilizing electronic components. The invention also relates to a disinfected electronic component comprising residues of the treatment medium and/or reaction products of the treatment medium and less than 2000 germs per m² on the surface of the disinfected electronic component, and to a sterilized electronic component comprising residues of the treatment medium and/or reaction products of the treatment medium and a germ-free surface.

### PRIOR ART

The peracetic acid-ethanol vacuum method is an established and cost-effective sterilization method for allogeneic bone transplants and also offers adequate antibacterial, antifungal and antiviral protection for allogeneic soft tissue transplants.

In the case of disinfection and/or sterilization by the use of PES in vacuo, the PES passes into the gas phase at a high vacuum even at a low temperature. This process is used to achieve the best possible disinfection and/or sterilization of the products at an innocuous temperature.

Introduced in the 1980s [Starke R, Hackensellner H A, von Versen R. Experimentelle Untersuchungen zur Entkeimung von Transplantationsmaterial mit Peressigsäure. Z Exp Chir Transplant Artificial organs. 1984; 17(5):254-8.], more than 60,000 allogeneic bone grafts have been sterilized with peracetic acid (PES, CH3CO3H) and ethanol to date [131]. Several studies have demonstrated sufficient efficacy and safety of the procedure and have not demonstrated any adverse effect on the structural and biomechanical properties of the transplants [132, 133]. Viral protection was also confirmed by the sterilization of soft tissue transplants such as Achilles tendon, cartilage and skin [152]. Furthermore, Scheffler et al. were not able to detect any impairment of the structural and material properties of human BPTB allografts after sterilization with the peracetic acid-ethanol vacuum method in an in vitro study [153]. In addition, PES showed no cytotoxic or pro-inflammatory effect on human patellar tendon allografts in another in vitro study [98. In this publication, the advantages of the peracetic acid/ethanol vacuum process are used for disinfecting and/or sterilizing electronic components.

### Object

It is therefore an object of the present invention to provide a method for disinfecting and/or sterilizing electronic components, with which electronic components can be disinfected and/or sterilized reliably and inexpensively.

It is also an object of the present invention to provide a device for disinfecting and/or sterilizing electronic components, with which electronic components can be disinfected and/or sterilized reliably and inexpensively. It is also an object of the present invention to provide a disinfected electronic component. It is also an object of the present invention to provide a sterilized electronic component.

The object is achieved by means of the method for disinfecting and/or sterilizing electronic components according to claim 1. Advantageous embodiments of the invention are provided in the dependent claims.

The method according to the invention for disinfecting and/or sterilizing electronic components has five method steps: In the first step, an electronic component is placed in a process chamber that can be evacuated. For the purposes of this specification, an electronic component is a component, system or product which has electrical components. It can be sealed, closed or encapsulated. Optionally, open electronic components can also be accessible. The process chamber can be evacuated and can be opened or closed in a gas-tight manner. The volume of the process chamber can be selected variably depending on the dimensions of the electronic component to be disinfected and/or sterilized. The placing of an electronic component can be carried out automatically, by remote control and/or by hand by a user. The process chamber can have suitable receptacles for this purpose.

The difference between a disinfection and a sterilization is that in a sterilizing treatment of an object all microorganisms, including their permanent forms (spores), are killed or irreversibly inactivated. The electronic component is then generally free of microorganisms capable of propagation; at most one of 10⁶ germs can survive, i.e. only one of 1,000,000 electronic components treated would not be sterile. Disinfectant treatment of an electronic component likewise kills or irreversibly inactivates pathogenic germs, but the reference to the number of germs to be eliminated is smaller by a power of 10 than during sterilization, the aim of disinfection being to reduce the germs by at least a factor of 10⁵.

In the second step, the process chamber is evacuated. A vacuum of less than 300 mbar, preferably less than 150 mbar, particularly preferably less than 50 mbar is preferably produced in the process chamber.

In the third step, a treatment medium is introduced. For the purposes of this document, a treatment medium is peracetic acid or a peracetic acid-containing mixture having a peracetic acid content of at least 2% by volume, preferably at least 3% by volume and particularly preferably at least 4% by volume of peracetic acid. Alternatively, the starting substances of the peracetic acid can also be introduced into the process chamber in order to allow the peracetic acid to be formed in situ.

In the fourth step, the disinfection and/or sterilization process is carried out. Optionally, the process parameters (pressure and temperature in the process chamber) are kept constant during an exposure time. This serves to eliminate germs, bacteria and viruses on the electronic component. Only the pressure is increased by the vaporization of the PES. Optionally, the pressure change can be avoided by appropriate pumping out.

In the fifth step, the pressure in the process chamber is increased. When the pressure is increased, the process chamber contains a concentration of the treatment medium which is below the concentration of the treatment medium in the process chamber during the holding time of the process. The concentration of the treatment medium during or after the increase of the pressure in the process chamber is by more than a factor of 10, particularly preferably by more than factor 30 below the concentration of the treatment medium during the holding time.

In a further development of the invention, the process chamber is preheated. In a further embodiment of the invention, the process chamber is preheated to the desired temperature of 35 °C-85 °C, preferably 40 °C-75 °C and particularly preferably 50 °C-65 °C. The method for disinfecting and/or sterilizing electronic components is thus accelerated.

In a further embodiment of the invention, the electronic components are packaged in sterilization bags before being placed in the process chamber. In a further aspect of the invention, the sterilization bags are permeable to vapors. Sterilization bags, also known as autoclave bags or laminated bags, are used to protect items from contamination. They are made of a material that withstands high temperatures and steam sterilization. This makes them ideal for use in hospitals, dental practices and other medical facilities. Sterilization bags provide a safe and efficient way to protect the electronic component from contamination. With their puncture-resistant construction and moisture-resistant material, they are ideal for use in any environment. Sterilization bags are puncture-resistant so that they are not damaged during the sterilization process. The material of the sterilization bag is moisture and bacteria resistant, so that the objects are protected from contamination. The sterilization bags are easy and simple to store so that they can be used in any environment.

In a further embodiment of the invention, the process chamber is closed after the electronic component has been placed in the process chamber. After closing, the process chamber is sealed in a gas-tight manner.

In a further embodiment of the invention, the process chamber is heated to process temperature after the process chamber has been closed. Due to the placement of the electronic component, the temperature in the process chamber decreases, so that it is reheated to a process temperature of 40 °C-65 °C, preferably 50 °C-55 °C.

In a further embodiment of the invention, the pressure in the process chamber is monitored. For this purpose, the process chamber is connected to a pressure sensor.

In an advantageous embodiment of the invention, the treatment medium is a PES solution. PES disinfects chemically and has an oxidizing effect on microorganisms. It has a broad spectrum of action, short exposure time and an irreversible effect. In addition, compared to other sterilization media, it is largely compatible with materials, can be added in exact amounts and, because of the low concentration used (dilution with water), is slightly to not damaging to the skin. Optionally, a mixture of peroxyacetic acid, acetic acid, a strong oxidizing agent such as, for example, hydrogen peroxide and water is used. The acids and hydrogen peroxide are powerful oxidizing agents that decompose microorganisms.

In a further embodiment of the invention, the process chamber is evacuated to a pressure of 1-50 mbar, preferably 10 mbar. In a further embodiment of the invention, the pressure in the process chamber is maintained below the boiling curve of the treatment medium after the introduction of the treatment medium. In a further development of the invention, the pressure is kept below the boiling curve of the treatment medium throughout the process duration of the disinfection and/or sterilization process. After the treatment medium has been introduced, the treatment medium passes immediately into the gas phase. As a result, even regions of the electronic component which are difficult to access are disinfected and/or sterilized.

In a further embodiment of the invention, after completion of the disinfection and/or sterilization process, the process chamber is flushed with ambient air, the flushing preferably being carried out at intervals. This ensures that the treatment medium has been completely removed from the process chamber and/or the electronic component and cannot escape uncontrollably when the process chamber is opened, so that the operator or a bystander does not inhale the solution when the door or access is opened. For the purposes of this specification, a ventilation medium is air and technical gases. In a further aspect of the invention, ambient air up to a chamber pressure of 200-500 mbar is introduced during flushing.

In a further embodiment of the invention, after introduction of the ambient air up to a pressure of 200-500 mbar, the process chamber is evacuated again to 1-100 mbar, preferably 10-40 mbar, particularly preferably 20 mbar. This likewise ensures that the treatment medium has been completely removed from the process chamber and cannot escape in uncontrolled manner when the process chamber is opened.

In a further embodiment of the invention, the steps according to claims 14 and 15 are repeated once, preferably three times and particularly preferably five times. This likewise ensures that the treatment medium has been completely removed from the process chamber and cannot escape uncontrolled when the process chamber is opened.

In a further embodiment of the invention, the pressure in the process chamber is increased to ambient pressure after completion of the flushing process. The disinfected products can then be removed from the process chamber.

In a further development of the invention, the treatment medium is discharged. The treatment medium is thus removed from the process chamber.

In a further embodiment of the invention, a neutralizing medium is introduced in order to neutralize the treatment medium. PES is an acid, therefore a base is optionally used as the neutralization medium. This likewise ensures that the treatment medium has been completely removed from the process chamber and cannot escape uncontrollably when the process chamber is opened.

The object is likewise achieved by means of the software program for carrying out the method according to one or more of claims 1 to 19.

The software program according to the invention is suitable for carrying out the process for controlling processes by means of defined values and/or curves related to pressure, injection quantities (with defined concentrations), temperature and PES concentration in the chamber and by means of material and/or product information, their quantity, size, shape, etc., their degree of contamination and loading and/or type of pollution (spores, viruses, etc.). The software program uses the hardware of the device for disinfecting and/or sterilizing electronic components.

The object is also achieved by means of the device for disinfecting and/or sterilizing electronic components. Advantageous embodiments of the invention are provided in the dependent claims.

The device according to the invention for disinfecting and/or sterilizing electronic components has an evacuatable process chamber. The process chamber can be evacuated and can be opened or closed in a gas-tight manner. The volume of the process chamber can be selected variably depending on the dimensions of the electronic component to be disinfected and/or sterilized.

The device also has a first connection and/or first reservoir for a treatment medium and a first inlet in the process chamber, wherein the first inlet is connected to a first connection and/or the first reservoir for the treatment medium. The treatment medium is preferably peracetic acid or a peracetic acid-containing mixture having a peracetic acid content of at least 2% by volume, preferably at least 3% by volume and particularly preferably at least 4% by volume of peracetic acid. Alternatively, the starting substances of the peracetic acid can also be introduced into the process chamber in order to allow the peracetic acid to be formed in situ.

The device likewise has a second connection and/or second reservoir for a ventilation medium and a second inlet in the process chamber, the second inlet being connected to the second connection and/or the second reservoir for the ventilation medium and/or to the first connection and/or the first reservoir. The ventilation medium is preferably air; technical gases, e.g. noble gases, are also possible.

Furthermore, the device has a pump, which is connected to one or more outlets, and a control for controlling the first inlet, the second inlet, the first connection, the second connection and/or the outlet. Optionally, the temperature control and/or the pressure and/or the injection quantity and/or the concentration of a treatment medium in the chamber is controlled by the control. Furthermore, the duration and the number of process phases and cycles can be controlled by the control.

The object is also achieved by means of the disinfected electronic component. An advantageous embodiment of the invention is provided in the dependent claim.

The disinfected electronic component has residues of the treatment medium and/or reaction products of the treatment medium of less than 2000 germs per m² on the surface of the disinfected electronic component. The disinfected electronic component according to the invention has a contamination with germs on its surface of less than 2000 germs per m², preferably of less than 1000 germs per m² and particularly preferably of less than 500 germs per m², which corresponds to a germ reduction of 84% to 99.9%. In a further development of the invention, the disinfected electronic component has residues of peracetic acid or residues of reaction products of peracetic acid.

The object is likewise achieved by means of the sterilized electronic component. An advantageous embodiment of the invention is provided in the dependent claim.

The sterilized electronic component has residues of the treatment medium and/or reaction products of the treatment medium and a germ-free surface. The sterilized electronic component according to the invention has a contamination with microbes on its surface of 0 if the probability of a surviving microbe is less than 1:1,000,000 (<=10⁻⁶ per unit of the electronic component). In a further development of the invention, the disinfected electronic component has residues of peracetic acid or residues of reaction products of peracetic acid. In an optional further development the concentration of the treatment medium and/or of reaction products of the treatment medium is below 0.1 ml/m² or below 0.32 mg/m² and in a particularly preferred embodiment below 0.05 ml/m² or below 0.16 mg/m².

Exemplary embodiments of the device according to the invention and of the method for disinfecting and/or sterilizing electronic components are shown schematically in the drawings in a simplified manner and are explained in more detail in the following description.

In the drawings:

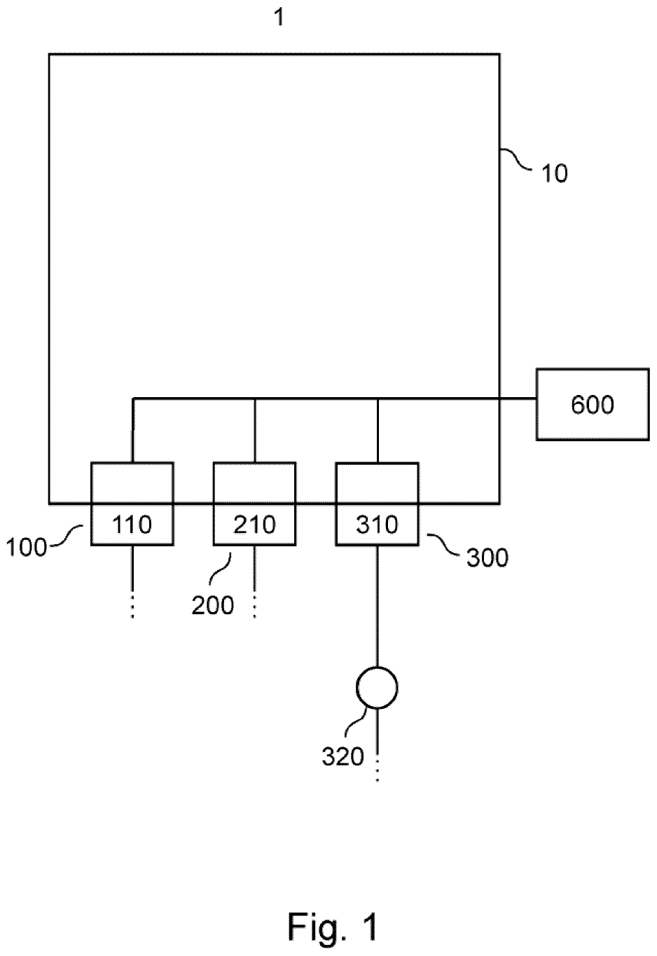

FIG. 1 shows a device for disinfecting and/or sterilizing electronic components

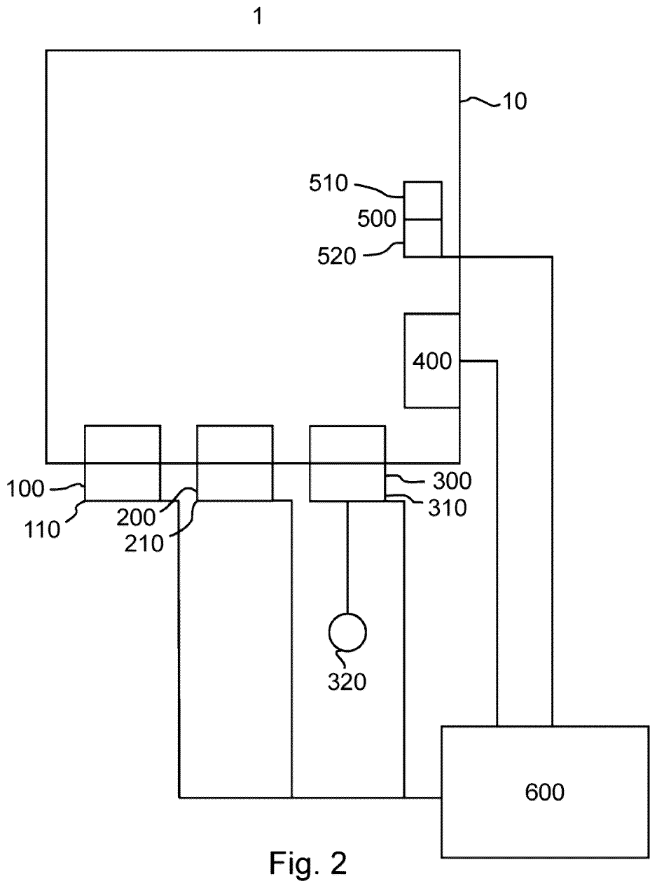

FIG. 2 shows a device for disinfecting and/or sterilizing electronic components, pressure and temperature sensors

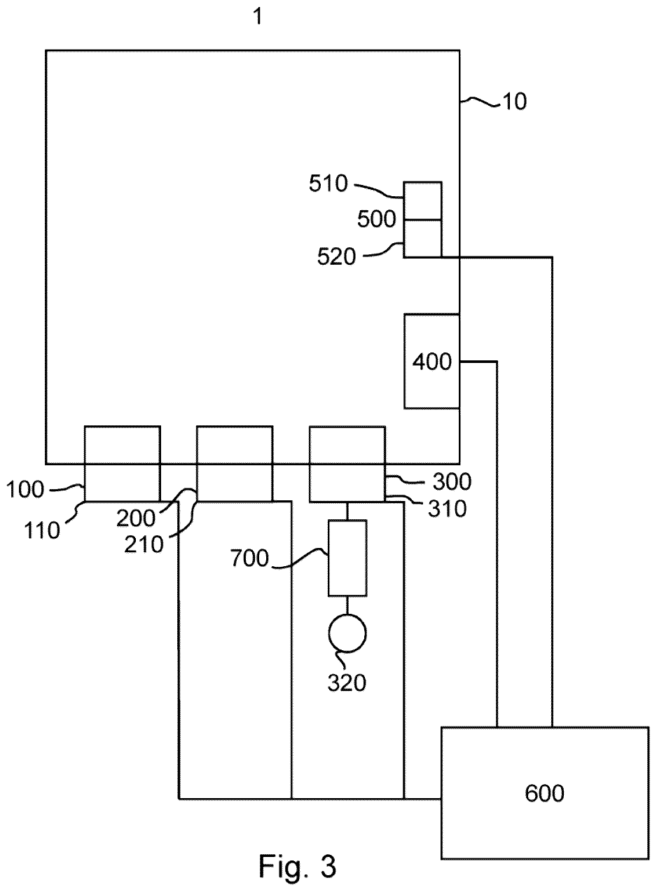

FIG. 3 shows a device for disinfecting and/or sterilizing electronic components, with HEPA-filter

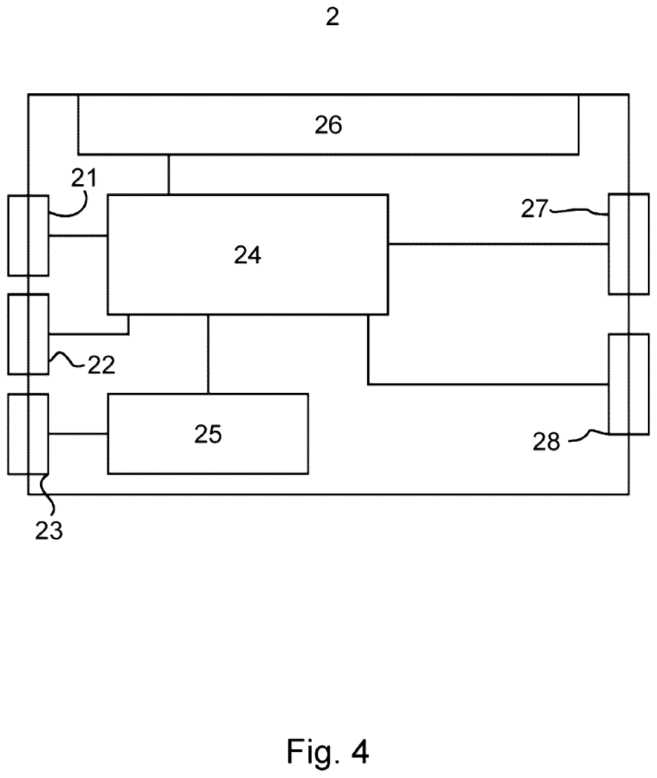

FIG. 4 shows an exemplary embodiment of an electronic component

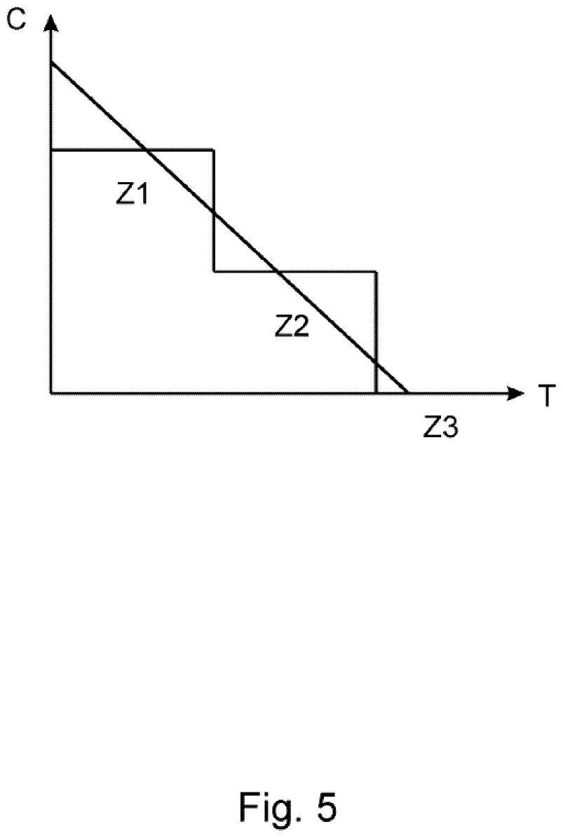

FIG. 5 shows an exemplary embodiment of the method for disinfecting and/or sterilizing electronic components

FIG. 1 shows an exemplary embodiment of the device 1 according to the invention for disinfecting and/or sterilizing electronic components. The device 1 has the process chamber 10, which is designed to be evacuable and has a volume of 50 liters, and 100 liters are also possible. For evacuation, the vacuum pump 320, designed as a rotary pump, is connected via the outlet connection 310 to the outlet 300 of the process chamber 10. The outlet 300 can be opened or closed and is connected to the control 600. In addition to a UI, the control 600 has a microcontroller and a memory which has a suitable software program for carrying out a disinfection and/or sterilization process of electronic components 2.

The process chamber 10 has a first inlet 100 which is connected via a first connection 110 to a reservoir for the treatment medium. The second inlet 200, which is connected via the second connection 210 to the reservoir for the ventilation medium, is also connected to the control 600. The inlets 100, 200, like the outlet 300, can each be opened or closed via valves, controlled by the control 600.

For disinfection and/or sterilization of electronic components 2 (see FIG. 4), the electronic component 2 is placed in the process chamber 10, for example in a suitable receptacle in the process chamber 10. The electronic component 2 is preferably packed in a steam-permeable sterilization bag before it is placed in the process chamber 10, in order not to contaminate the electronic component 2 during and after the disinfection and/or sterilization and to be able to act simply and safely in the decontamination process on the contaminated electronic component.

The process chamber 10 is then sealed in a gas-tight manner and evacuated to 20 mbar by means of the vacuum pump 320. The treatment medium is then introduced by the control 600 sending a signal for opening the first inlet 100 to the first inlet 100. The first inlet 200 is connected to the reservoir for the treatment medium. In all the exemplary embodiments shown here, the treatment medium is a mixture of peroxyacetic acid, water and, if appropriate, other constituents. The treatment medium is preferably peracetic acid or a peracetic acid-containing mixture having a peracetic acid content of at least 2% by volume, preferably at least 3% by volume and particularly preferably at least 4% by volume of peracetic acid. Alternatively, the starting substances of the peracetic acid can also be introduced into the process chamber in order to allow the peracetic acid to be formed in situ.

Due to the low pressure in the process chamber 10, the solution is brought into the gaseous state in order to fill the process chamber 10 and carry out the oxidation process on the objects to be disinfected. Different concentrations of PES are provided for the process in order to be able to flexibly design the disinfection and/or sterilization process. The first inlet 100 is closed again after introduction of the treatment medium.

For the disinfection and/or sterilization of electronic components 2, the electronic component 2 is left in the process chamber 10 for between 5 minutes and 120 minutes at constant pressure and temperature in the process chamber 10 and is exposed to the PES atmosphere. Thereafter, the pressure in the process chamber 10 is increased by the control 600 sending a signal for opening the second inlet 200 to the second inlet 200. The second inlet 200 is connected to the reservoir for the treatment medium. In all exemplary embodiments, the ventilation medium is air, and technical gases, for example noble gases, are also possible. When the pressure is increased, the process chamber contains a concentration of the treatment medium which is below the concentration of the treatment medium in the process chamber during the holding time of the process. The concentration of the treatment medium during or after the increase of the pressure in the process chamber is by more than a factor of 10, particularly preferably by more than factor of 30 below the concentration of the treatment medium during the holding time. The process chamber 10 can be opened and the electronic component 2 removed.

Further exemplary embodiments of the device 1 according to the invention for disinfecting and/or sterilizing electronic components are shown in FIGS. 2 and 3. The devices 1 shown here correspond to those in the preceding exemplary embodiment (cf. FIG. 1), only the process chamber 10 has a heating device 400, which is designed as a resistance heating and is connected to the control 600. In addition, a sensor unit 500 with a temperature sensor 510 and a pressure sensor 520 is arranged in the process chamber 10 and is likewise connected to the control 600 (FIG. 2). In a further embodiment, the device 1 has a HEPA filter 700 which is arranged in the line between pump 320 and outlet 300 (FIG. 3).

For the disinfection and/or sterilization of electronic components 2, after placing the electronic component 2 in the process chamber 10 and evacuating it, the temperature in the process chamber 10 is increased by means of the heating device 400 after closing the process chamber 10 to a temperature of 50 °C to 55 °C, or alternatively to a temperature of 40 °C to 65 °C. In order to shorten the heating process, the process chamber 10 can be preheated before the electronic component 2 is placed in the process chamber 10.

In the next step, the treatment medium (PES solution) is introduced into the process chamber 10 via the first inlet 100. In an advantageous further development of the invention, the injection quantity and/or the concentration of a treatment medium in the process chamber 10 is controlled with the aid of the control 600. Furthermore, the duration and the number of process phases and cycles are controlled. The PES solution begins to boil immediately and the pressure in the process chamber 10 increases. During part of the process, the control 600 controls the pressure and temperature in the process chamber 10 in such a way that the boiling curve of the PES solution is at least partially not exceeded in order to prevent the PES solution from condensing out. This state is kept constant for part of the process duration. This serves the elimination of germs, bacteria and viruses on the electronic component 2. Only the pressure in the process chamber 10 is increased by the evaporation of the PES. Optionally, this pressure change can be avoided by appropriate pumping by means of the pump 320. By means of the temperature sensor 510, the control 600 continuously detects the temperature in the process chamber 10 during the process and controls the temperature in the process chamber 10 via the heating device 400. The pressure in the process chamber 10 is detected by the pressure sensor 520 and is likewise regulated by the control 600, in that either the inlet 100 is opened or the pressure in the chamber is reduced via the outlet 300 and the pump 320.

After the end of the process, the ventilation medium (air) is flushed through the process chamber 10 at intervals. The ventilation medium is preferably germ-free, i.e. no microorganisms and/or viruses which are greater than 0.45 micrometer, preferably 0.22 micrometer, are contained in the medium. For this purpose, ambient air is allowed into the process chamber 10 up to a pressure in the process chamber 10 of 200 mbar to 500 mbar, preferably 300 mbar, via the second inlet 200. The mixture is then pumped off at 1 mbar to 100 mbar, preferably about 40 mbar, particularly preferably about 20 mbar. This flushing process is carried out one to five times, preferably three times, in order to ensure that the treatment medium has been completely removed from the process chamber 10 and cannot escape uncontrollably when the process chamber 10 is opened, and that the operator or a bystander does not inhale the solution when the door or the access is opened. In a further development, the ventilation medium is introduced until the concentration of the treatment medium in the chamber is between 0.5 g/m and 4 g/m³; preferably between 1 g/m³ and 3 g/m³; particularly preferably between 1.5 g/m³ and 2.5 g/m³. The air inlet or the air discharge via an outlet 300 preferably takes place via HEPA filters. The HEPA filter 700 serves to protect the environment and persons from germs during the generation of vacuum at the beginning of the process and also to protect the electronic component 2 from contamination. Optionally, a neutralizing medium, e.g. a gaseous base, can be introduced before the flushing process with the ventilation medium in order to neutralize the treatment medium.

FIG. 4 shows an electronic component 2 which can be disinfected and/or sterilized by means of the method according to the invention and the device 1 according to the invention (cf. FIGS. 1 to 3). In this exemplary embodiment, the electronic component 2 is prototypical for a commercially available smartphone 2. The smartphone 2 has a first connection 21 for headphones, a second connection 22 for a USB cable and a third connection 23 for a charging cable. The first connection 21 and the second connection 22 are connected to the circuit board 24, and the third connection 23 is connected to the power supply 25 (battery), which is also connected to the circuit board 24. The circuit board 24 is also connected to the display 26, the microphone 27 and the loudspeaker 28. The aforementioned components of the smartphone 2 are arranged in a housing.

After carrying out the process with the device 1 according to the invention, the electronic component 2 has traces of the treatment medium (PES) and/or of reaction products of the treatment medium with other substances on its surface. In an optional further development the concentration of the treatment medium and/or of reaction products of the treatment medium is below 0.1 ml/m² or below 0.32 mg/m² and in a particularly preferred embodiment below 0.05 ml/m² or below 0.16 mg/m².

The disinfected electronic component 2 according to the invention, after carrying out the method with the device 1 according to the invention, has a contamination with germs on its surface of less than 2000 germs per m², preferably of less than 1000 germs per m² and particularly preferably of less than 500 germs per m² surface, which corresponds to a germ reduction of 84% to 99.9%. The sterilized electronic component 2 according to the invention has a contamination with microbes on its surface of 0 if the probability of a surviving germ is less than 1:1,000,000 (<=10⁻⁶ per unit of the electronic component 2).

The method according to the invention, which can be carried out by means of the device 1 according to the invention, is effective in such a way that even the inner surfaces of the electronic component 2 protected by the housing, which is splashproof in this exemplary embodiment, have such low contamination with germs as the outer surfaces after carrying out the method.

FIG. 5 shows an exemplary embodiment of the method according to the invention for disinfecting and/or sterilizing electronic components 2, which is carried out in three cycles. The graph shows the concentration of spores/viruses/bacteria on the inner and outer surfaces of the electronic component 2 (ordinates) over time (abscissa).

The disinfection and sterilization process is usually carried out depending on the product to be sterilized/disinfected, the framework conditions and predetermined requirements according to processes optimized especially in the control 600 and the process. For this purpose, different cycles, intervals and process parameters can be set via a software program stored in the control 600 or, if required, also individually via the UI of the control 600. For example, different cycles of disinfection and/or sterilization may have to be carried out at a high germ load of the electronic component 2, since the PES solution is consumed at a high germ load, so that the subsequent addition of PES may be necessary. This step will take place, among other things, in cycles. During the process, the treatment medium (PES) is pumped out and added or ventilated. In addition, the course of the cycle is also suitable for small volumes both of the process chamber 10 and of the electronic component 2 as well as difficult to reach and complex geometries of the electronic component 2.

TABLE 1
Overview of validation tests for disinfection
of spore strips SAL 10⁵

| | PES in | Concentration PES | Process | |
|---|---|---|---|---|
| | solution | in process chamber | duration | Biological result #not |
| | [%] | [g/m³] | [min] | sterile/#tested |
| 1 | 6 | 3 | 240 | 0/2 |
| 2 | | | 120 | 0/1 |
| 3 | | 1.7 | 150 | 0/2 |
| 4 | 4 | 2 | 120 | 0/1 |
| 5 | | 1.9 | 120 | 0/2 |
| 6 | | | 90 | 0/2 |
| 7 | | | 60 | 0/4 |
| 8 | | | 45 | 0/1 |
| 9 | | | 40 | 0/2 |
| 10 | | | 30 | 0/2 |
| 11 | | | 20 | 0/2 |
| 12 | | | 15 | 0/1 |
| 13 | | | 10 | 0/1 |
| 14 | | | 5 | 0/1 |
| 15 | | 1.7 | 20 | 0/1 |
| 16 | | | 15 | 0/1 |
| 17 | | 1.5 | 240 | 0/2 |
| 18 | | | 150 | 0/2 |
| 19 | | 1.3 | 20 | 0/1 |
| 20 | | | 15 | 0/1 |
| 21 | | 0.94 | 60 | 0/5 |
| 22 | | | 40 | 0/1 |
| 23 | | | 30 | 0/2 |
| 24 | | | 20 | 0/2 |
| 25 | | | 15 | 0/1 |
| 26 | | | 10 | 0/1 |
| 27 | | | 5 | 0/1 |
| 28 | 4 | 0.47 | 60 | 0/1 |
| 29 | | | 30 | 0/1 |
| 30 | | | 15 | 0/1 |
| 31 | | | 10 | 0/1 |
| 32 | | | 5 | 0/1 |

TABLE 2
Overview of validation tests for disinfection of laboratory
utensils dripped with spore suspension SAL 10⁶

| | PES in | Concentration PES | Process | |
|---|---|---|---|---|
| | solution | in process chamber | duration | Biological result #not |
| | [%] | [g/m³] | [min] | sterile/#tested |
| 5 | 4 | 1.9 | 120 | 0/4 |
| 7 | | | 60 | 0/4 |
| 8 | | | 45 | 0/4 |
| 12 | | | 15 | 0/4 |
| 13 | | | 10 | 0/4 |
| 14 | | | 5 | 0/4 |
| 15 | | 1.7 | 20 | 0/4 |
| 16 | | | 15 | 0/4 |
| 19 | | 1.3 | 20 | 0/4 |
| 20 | | | 15 | 0/4 |
| 21 | | 0.94 | 60 | 0/4 |
| 22 | | | 40 | 0/4 |
| 23 | | | 30 | (pipette) 1/8 |
| 24 | | | 20 | 0/8 |
| 25 | | | 15 | (pipette) 1/4 |
| 26 | | | 10 | 0/4 |
| 27 | | | 5 | (pipette) 1/4 |
| 28 | 4 | 0.47 | 60 | 0/4 |
| 29 | | | 30 | 0/4 |
| 30 | | | 15 | (Eppi) 1/4 |
| 31 | | | 10 | (pipette) 1/4 |
| 32 | | | 5 | (mask, pipette) 2/4 |

Disinfectant treatment of an object likewise kills or irreversibly inactivates pathogenic germs, but the reference to the number of germs to be eliminated is smaller by a power of 10 than during sterilization, the aim of disinfection being to reduce the germs by at least a factor of 10⁵. The disinfecting treatment then causes the object to cease to be infectious.

The efficiency of the disinfecting treatment is defined by the probability of the presence of microorganisms hostile to life. This probability is expressed by the sterility assurance level (SAL), a SAL of at least 10⁻⁵ defines disinfection, a SAL of at least 10⁻⁶ sterilization, i.e. the lower the SAL value, the higher the safety.

The disinfecting and sterilizing agent acting in the device presented here is peracetic acid (PES) in a solution diluted with water. PES disinfects chemically and has an oxidizing effect on microorganisms and/or viruses. It has a broad spectrum of action, short exposure time and an irreversible effect. In addition, compared to other sterilization media, it is largely compatible with materials, can be added in exact amounts and, because of the low concentration used (dilution with water), is slightly to not damaging to the skin.

The validation tests of the “disinfection and sterilization device” were carried out with spores of the bacterium Geobacillus stearothermophilis. Bacterial spores have a very high resistance (C) to chemical disinfection and/or sterilization. If bacterial spores can be successfully disinfected in the processes, it is to be assumed that the method also successfully disinfects microorganisms with moderate resistance (A) such as lipophilic viruses, vegetative bacteria, fungi (including spores), leading organisms such as E. faecium, S. aureus, P. aeruginosa, A. niger and microorganisms with high resistance (B) such as mycobacteria, hepatitis B virus and hydrophilic viruses such as the leading virus polio.

The validation experiments were first carried out with bio-indicator-spore strips 6×36 mm, on which in each case a colony-forming unit (CFU) of 10⁵ of the Geobacillus stearothermophilus was inoculated. The spore strips were each packaged in a sterilization bag. Different process times and different concentrations of PES were tested in the experiments. All tests were carried out at a chamber temperature of 50-55 °C.

No subsequent growth or multiplication of the spores could be detected in all the spore strips tested at different PES concentrations and different process times.

In addition, untreated test samples were added to each procedure. This ensured that the samples were inoculated with spores.

In addition to the validation tests with the SAL 10⁵ spore strips, various disposable products were also drizzled with a spore suspension. These were a vaccine eye, a disposable 3 ml pipette, a microreaction vessel with a lid (Eppi) and a piece of nose-mask. The spore suspension was an alcoholic solution interspersed with Geobacillus stearothermophilus at a CFU/ml of at least 10⁶. Due to the small amount, the samples were each dripped with 2 drops of the suspension (in the pipette and the Eppi the suspension was dripped into this) and then placed in a sterilization bag. One drop corresponds to 0.05 ml, so two drops correspond to a unit of 0.1 ml. Therefore the CFU per object is about 10⁵.

The samples were co-tested with some of the spore strip tests listed above. After the treatment, the swabs of the samples, analogous to the spore strips, were placed in a nutrient solution in the external laboratory for seven days and then examined for possible growth of the spores.

In various experiments, spore growth was observed after seven days. This is due to the relatively low PES concentration in the process chamber as well as to the short process duration.

The disinfection or sterilization system according to the invention is designed in such a way that selectively different concentrations of PES in the vacuum of the process chamber can be set. By means of the combination of special injection technology, adapted vacuum process and process cycles, it is ensured that a distribution of the disinfectant and/or sterilizing agent mixture also takes place reliably at all inaccessible points and thus the PES has a very high activity against biogenic impurities even at low temperatures of 40 °C to 65 °C, preferably 50 °C to 55 °C.

The disinfection or sterilization system according to the invention, including the disinfection and/or sterilization processes and associated equipment technology, thus has the following decisive advantages: high efficiency, low-temperature processes (also suitable for thermolabile polymers), low operating costs, short process times, individual adaptations of the disinfection and/or sterilization programs to different requirements, very good scalability (from mobile desktop equipment to mobile room-sized systems) and general use of a reliable process which is not susceptible to malfunctions.

Due to the combination of the above advantages this innovative disinfection and sterilization system—in contrast to the methods already used—is therefore excellently suited for numerous known and new fields of application. For example, infection protection in small to large healthcare and care facilities, where the entire spectrum from compact desktop devices to large systems is required. The same applies to the areas of application of fire brigades and civil protection, in which the systems and equipment must be particularly robust and reliable as well as in some cases mobile. In future, the PES processing system will include the following process-specific and application-related innovations in particular:
a. Novel, very widely usable low-temperature chemical disinfection and/or sterilization system
   The PES as reactive component can either be produced in low concentrations in the in-situ mixing of the different starting materials or can be adjusted to higher concentrations by selective mixing from a storage container in the system. PES reacts with the bacteria, viruses, spores, etc., which are killed quickly and efficiently. The individual components (hydrogen peroxide and acetic acid) for the in-situ production of PES are low-cost chemicals available worldwide, which can be handled safely while taking into account the necessary occupational safety requirements.
   Gaseous PES is characterized by a high sterilizing effect. The use of gaseous PES in an automated vacuum process of a closed system ensures both a hazard-minimized handling and a safe effect
   Standardized, validatable method (no manual disinfection, e.g. wipe disinfection)
   The starting materials (hydrogen peroxide and acetic acid) can be removed quantitatively again from the treatment system and treated products very easily) so that the treated protective clothing can be reused without endangering the operators.
b. Mobile, tailor-made disinfection and/or sterilization device technology can be used for almost all application and use conditions for rapid PES disinfection and/or sterilization even on site in the event of major damage events.
c. Integrated monitoring and documentation system for the disinfection and/or sterilization process, i.e. for the protective effect after treatment of the PPE
d. Special design and process control of this chemical-physical preparation process (PES, vacuum, temperature) in combination with the specially adapted processing system provides short treatment times of in some cases 8-25 minutes. These treatment times are dependent on the treatment objective (disinfection to sterilization if necessary), the PPE materials or material combinations as well as the surfaces and geometries.
e. The disinfection and/or sterilization of the PPE is possible in the new preparation system in the sterilization bag and thus safe and contamination-free handling.

### LIST OF REFERENCE NUMERALS

1 device for disinfecting and/or sterilizing electronic components
10 process chamber
100 first inlet
110 first connection
200 second inlet
210 second connection
300 outlet
310 outlet connection
320 pump
400 heating device
500 sensor unit
510 temperature sensor
520 pressure sensor
600 control
700 HEPA filter
2 electronic component
21 first connection
22 second connection
23 third connection
24 circuit board
25 power/battery
26 display
27 microphone
28 loudspeaker

1. A method for disinfecting and/or sterilizing electronic components (2) comprising the steps:
placing an electronic component (2) in a process chamber (10) that can be evacuated,
evacuating the process chamber (10),
introducing a treatment medium,
carrying out the disinfection and/or sterilization process,
increasing the pressure in the process chamber (10).

2. The method for disinfecting and/or sterilizing electronic components (2) according to claim 1,
characterized in that
the process chamber (10) is preheated.

3. The method for disinfecting and/or sterilizing electronic components (2) according to claim 2,
characterized in that
the process chamber (10) is preheated to the desired temperature of 35 °C-85 °C, preferably 40 °C-75 °C and particularly preferably 50 °C-65 °C.

4. The method for disinfecting and/or sterilizing electronic components according to claim 1,
characterized in that
the electronic components (2) are packaged in sterilization bags before being placed in the process chamber (10).

5. The method for disinfecting and/or sterilizing electronic components (2) according to claim 4,
characterized in that
the sterilization bags are permeable to vapors.

6. The method for disinfecting and/or sterilizing electronic components (2) according to claim 1,
characterized in that
the process chamber (10) is closed after the electronic components (2) have been placed in the process chamber (10).

7. The method for disinfecting and/or sterilizing electronic components (2) according to claim 1,
characterized in that
the process chamber (10) is heated to process temperature after the process chamber (10) has been closed.

8. The method for disinfecting and/or sterilizing electronic components (2) according to claim 1,
characterized in that
the pressure in the process chamber (10) is monitored.

9. The method for disinfecting and/or sterilizing electronic components (2) according to claim 1,
characterized in that
the treatment medium is a PES solution or a PES-containing solution.

10. The method for disinfecting and/or sterilizing electronic components (2) according to claim 1,
characterized in that
the process chamber (10) is evacuated to a pressure of 1-300 mbar, preferably 10-250 mbar and particularly preferably 10-50 mbar.

11. The method for disinfecting and/or sterilizing electronic components (2) according to claim 1,
characterized in that
the pressure after introduction of the treatment medium is kept partly below the boiling curve of the treatment medium.

12. The method for disinfecting and/or sterilizing electronic components (2) according to claim 11,
characterized in that
the pressure is kept below the boiling curve of the treatment medium for part of the process duration of the disinfection and/or sterilization process.

13. The method for disinfecting and/or sterilizing electronic components (2) according to claim 1,
characterized in that
after completion of the disinfection and/or sterilization process, the process chamber (10) is flushed with ambient air,
wherein the flushing is preferably performed at intervals.

14. The method for disinfecting and/or sterilizing electronic components (2) according to claim 13,
characterized in that
ambient air up to a chamber pressure of 200-800 mbar, preferably 200-700 mbar and particularly preferably 200-500 mbar is introduced during flushing.

15. The method for disinfecting and/or sterilizing electronic components (2) according to claim 14,
characterized in that
after introduction of the ambient air up to a pressure, the process chamber (10) is evacuated again to 1-500 mbar, preferably 10-400 mbar, particularly preferably 50-250 mbar.

16. The method for disinfecting and/or sterilizing electronic components (2) according to claim 13,
characterized in that
the steps according to claims 14 and 15 are repeated once, preferably three times and particularly preferably five times.

17. The method for disinfecting and/or sterilizing electronic components (2) according to claim 1,
characterized in that
after completion of the flushing process, the pressure in the process chamber (10) is increased to ambient pressure.

18. The method for disinfecting and/or sterilizing electronic components (2) according to claim 1,
characterized in that
the treatment medium is discharged.

19. The method for disinfecting and/or sterilizing electronic components (2) according to claim 1,
characterized in that
a neutralizing medium is introduced to neutralize the treatment medium.

20. A software program for carrying out the method according to claim 1.

21. A device (1) for disinfecting and/or sterilizing electronic components (2), comprising
a process chamber (10) that can be evacuated,
a first connection (110) and/or first reservoir for a treatment medium
a first inlet (100) in the process chamber (10); wherein the first inlet (100) is connected to a first connection (110) and/or the first reservoir for the treatment medium
a second connection (210) and/or second reservoir for a ventilation medium
a second inlet (200) in the process chamber (10), wherein the second inlet (200) is connected to the second connection (210) and/or the second reservoir for the ventilation medium and/or the first connection (110) and/or the first reservoir.
a pump (320) connected to one or more outlets (300)
a control (600) for controlling the first inlet (100), the second inlet (200), the first connection (110), the second connection (210) and/or the outlet (300).

22. A disinfected electronic component (2) comprising residues of the treatment medium and/or reaction products of the treatment medium with fewer than 2000 germs per m² on the surface of the disinfected electronic component (2).

23. The disinfected electronic component (2) according to claim 22,
characterized in that
the disinfected electronic component (2) contains residues of peracetic acid or residues of reaction products of peracetic acid.

24. A sterilized electronic component (2) with residues of the treatment medium and/or reaction products of the treatment medium and a germ-free surface.

25. The sterilized electronic component (2) according to claim 24,
characterized in that
the sterilized electronic component (2) contains residues of peracetic acid or residues of reaction products of peracetic acid.
